SEQUENCE LISTING (4 sequences)

SEQ ID NO: 1 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 2 - protein, 20 aa, Homo sapiens
RGASQAGAPQGRVPEARPNS

SEQ ID NO: 3 - DNA, 26 bp, Artificial Sequence
  Synthetic primer
ggatccccatggctccgcaccgcccc

SEQ ID NO: 4 - DNA, 27 bp, Artificial Sequence
  Synthetic primer
aagctttcaggcagccagctcagccat

(19) United States
(12) Patent Application Publication
(10) Pub. No.: US 2013/0266970 A1
(43) Pub. Date: Oct. 10, 2013

(54) PLASMA BIOMARKER FOR DISTAL METASTASIS IN COLORECTAL CANCER

(71) Applicant: CHANG GUNG UNIVERSITY, Tao-Yuan (TW)

(72) Inventors: Jau-Song YU, Tao-Yuan (TW); Ming-Hung TSAI, Tao-Yuan (TW); Ling-Ling Hsieh, Tao-Yuan (TW)

(73) Assignee: CHANG GUNG UNIVERSITY, Tao-Yuan (TW)

(21) Appl. No.: 13/651,186

(22) Filed: Oct. 12, 2012

(30) Foreign Application Priority Data
Apr. 10, 2012 (TW) 101112629

(51) Int. Cl. G01N 33/68

(52) U.S. Cl. CPC G01N 33/6893
USPC 435/7.92; 530/350; 436/501; 435/7.1; 530/387.9

(57) ABSTRACT

The present invention proposes secretory gelsolin (pGSN) as a plasma biomarker for evaluating distal metastasis of colorectal cancer. The present invention uses a special amino acid sequence of pGSN to fabricate a pGSN-specific polyclonal antibody. The present invention further uses the pGSN-specific polyclonal antibody to develop a high-specificity ELISA method and an assay kit thereof for evaluating distal metastasis of colorectal cancer.

### BACKGROUND OF THE INVENTION

1. Field of the Invention

The present invention relates to a biomarker for detecting distal metastasis in colorectal cancer, particularly to a secretory gelsolin (pGSN) biomarker for detecting distal metastasis in colorectal cancer.

2. Description of the Related Art

Under influence of the western diet style, colorectal cancer (CRC) has been the third most common cause of cancer mortality in Taiwan, as well as in the world. Distal metastasis is one of the primary causes of death induced by cancers, including CRC (Refer to Gupta, G. P. and Massague, J., 2006, Cell 127: 679-695; Mehlen, P. and Puisieux, A., 2006, Nat Rev Cancer 6: 449-458). Surgery is the mainstay for treatment of CRC. However, more than half of all CRC patients will develop metastases. Among CRC patients with metastases, the 5-year survival rate may be lower than 15%. Therefore, distal metastasis is one of the primary causes of CRC-induced death.

Surgery can promote the 5-year survival rate of CRC patients if distal metastasis is detected earlier and confined locally. The imaging inspection is the main clinical measure to diagnose distal CRC metastasis, including CT scans, bone scans, and positron emission tomography (PET). The patients needing image inspection may be classified into two groups. One group includes the patients who are newly diagnosed to have CRC, and image inspection is to examine whether distal metastasis has occurred. The other group includes Stage I-III CRC patients who have been treated with surgery and are suspect to have distal metastasis because their carnociembryonic antigen (CEA) increases abruptly in the routine post-operation tracking inspection, and imaging inspection is to verify the distal metastasis.

CEA is the primary blood biomarker that assists in imaging inspections (such as CT scans, bone scans, and PET) to evaluate the existence and recurrence (including distal metastasis) of CRC. For the abovementioned second group of patients, whether to be further inspected with imaging is dependent on the CEA level detected in the post-operation tracking inspections. After cancer has been removed with surgery, CEA decreases to the normal range. However, recurrence will cause CEA to increase (Refer to Tan, E. et al. 2009, Surg Oncol 18: 15-24). For detecting metastasis in CRC patients, the sensitivity of CEA exceeds 70% for distal liver metastasis but is lower than 50% for lung metastasis (Refer to Moertel, C. G. et al. 1993, JAMA 270: 943-947). Therefore, the sensitivity of CEA in evaluating distal CRC metastasis is still insufficient (only about 50-70%).

Thus, we need additional blood biomarkers cooperating with CEA to promote the efficiency and sensitivity of detection distal CRC metastasis. Many medical research reports have pointed out that the tumor biomarker panel consisting of several tumor biomarkers can obviously improve the specificity and sensitivity of detect malignant tumors (Refer to Conrads, T. P. et al. 2003, Expert Rev Mol Diagn 3: 411-420; Mor, G et al. 2005, Proc Natl Acad Sci USA 102: 7677-7682; Xiao, T. et al. 2005, Mol Cell Proteomics 4:1480-1486; Polanski and Anderson, 2007, Biomark Insights 1: 1-48). Therefore, it has been one of the primary subjects in CRC-related research to find and verify novel and effective blood biomarkers to assist CEA in detecting distal CRC metastasis.

At present, some scholars declare that they have found some biomarker proteins associated with distal CRC metastasis. However, most of these biomarkers are tissue biomarkers existing in tumor tissues. These tissue biomarkers do express abnormally in tumor tissues. However, whether these biomarkers can be detected in blood and whether their levels vary in blood are still uncertain, not to mention detecting these biomarkers in blood (serum or plasma) samples clinically. It has been reported by several research papers that EC39, amphiregulin and dUTPase are associated with distal metastasis based on immunohistochemical approach (Refer to Yoshikawa, R. et al. 2006, World J Gastroenterol 12: 5884-5889; Yamada, M. et al. 2008, Clin Cancer Res 14: 2351-2356; Kawahara, A. et al. 2009, J Clin Pathol 62: 364-369). However, very few reports have sought to search for CRC metastasis-associated biomarkers in blood samples.

Proteomics-based technologies, which are powerful tools for the global and comprehensive analysis of biological specimens, have been widely applied for cancer biomarker discovery (Refer to Zhao, Y. et al. 2009, Expert Rev Proteomics 6: 115-118; Hung, K. E. et al. 2010, Gastroenterology 138: 46-51). In the context of CRC, numerous proteomics studies have sought to identify potential biomarkers for CRC diagnosis and/or prognosis using tissue specimens or cancer cell lines as the study materials. So far, however, little effort has been made to unravel blood biomarkers for detecting CRC metastasis using proteomic methodologies. The discovery of protein biomarkers in blood samples is complicated by the broad dynamic range of proteins present in serum/plasma and the diversity across clinical specimens. The depletion of abundant proteins, the fractionation of samples by multi-dimensional separation, and the use of pooled samples may represent feasible solutions to these problems.

The present invention is devoted to searching for novel plasma biomarkers, and investigating the role the related biomarker in distal CRC metastasis. The present invention proposes a secretory gelsolin (pGSN), which is a protein secreted to blood, and which has not yet been used as a blood biomarker for detecting distal CRC metastasis so far. The present invention further uses a special amino acid sequence of pGSN to fabricate a pGSN-specific polyclonal antibody, which can be applied to early detection and prevention of distal metastasis of cancer.

### SUMMARY OF THE INVENTION

The present invention is based on the following discoveries: when the colorectal cancer (CRC) has progressed to the stage of distal metastasis, the secretory pGSN, i.e. plasma gelsolin (pGSN), in patient's plasma increases significantly; using both pGSN and CEA (carcinoembryonic antigen) can detect distal CRC metastasis more accurately than only using CEA; the fact that pGSN levels in the plasma of stage IV patients is significantly higher than those in stage I-III patients, indicating that pGSN may apply to detection and early diagnosis of distal CRC metastasis.

In one aspect, the present invention proposes a plasma biomarker for determining distal metastasis of CRC, which comprises at least one pGSN. In some embodiments of the present invention, the plasma biomarker further comprises at least one existing plasma biomarker for determining distal metastasis of CRC, which includes CEA.

In some embodiments, the abovementioned plasma biomarker is applied to determining distal CRC metastasis in cooperation with an enzyme-linked immunosorbent assay (ELISA), a bead-based immunoassay, a mass spectrometry-based assay, or a combination of mass spectrometry-based assay and immunoassay.

In one aspect, the present invention proposes a method for detecting distal CRC metastasis, which comprises a sampling step: obtaining a blood sample from a testee; a detection step: detecting at least one plasma biomarker in the blood sample, including pGSN; and an analysis step: calculating the concentration of the plasma biomarker in the blood sample with a standard curve, and comparing the concentration of the plasma biomarker of the blood sample with that of a healthy person. In some embodiments, the abovementioned pGSN has an amino acid sequence of SEQ ID NO:1 or an amino acid sequence 95% identical to the amino acid sequence of SEQ ID NO:1.

In some embodiments, the abovementioned method further comprises a step: detecting an existing distal CRC metastasis plasma biomarker, which includes CEA.

In some embodiments, the blood sample is a whole blood sample, a blood serum sample, or a plasma sample.

In one aspect, the present invention proposes a kit for detecting distal CRC metastasis, which comprises a pGSN-specific antibody. In some embodiments, the pGSN-specific antibody combines with a protein containing an amino acid sequence SEQ ID NO:1.

In some embodiments, the abovementioned pGSN-specific antibody is an antibody specific to a peptide antigen containing an amino acid sequence of SEQ ID NO:2. In some embodiments, the antibody of the present invention is a monoclonal antibody, a polyclonal antibody or a single chain antibody.

### BRIEF DESCRIPTION OF THE DRAWINGS

FIG. 1 shows a flowchart of the strategy of using the proteomic technology to search for CRC metastasis-associated biomarkers in the blood samples of CRC patients.

FIG. 2 shows the fractionation, visualization and quantitative analysis of the Cy-dye labeled plasma proteins. FIG. 2 (A) Samples form ET and LT in Exp. 1 and Exp. 2 were labeled by Cy3 and Cy5, respectively. The Cy-dye-labeled proteins were fractionated by anion-exchange chromatography using a fast protein liquid chromatography (FPLC). FIG. 2 (B) shows the log Cy5/Cy3 ratio obtained in Exp. 1 and Exp. 2. All protein bands in the Cy3 and Cy5 images obtained from the Typhoon 9400 scanner were analyzed using the ImageQuant V5.2 software (GE Healthcare).

FIG. 3 shows the representative fluorescent images of proteins differentially expressing in Exp 1 and Exp 2, wherein the ratios of Cy5 to Cy3 are shown on the bottoms of the fluorescent images, and wherein the numbers of protein band signals and electrophoresis films are shown on the right sides of the fluorescent images.

FIG. 4 shows that the pGSN concentration increases in plasma with CRC metastasis. The top illustration of FIG. 4A shows the representative fluorescent images of pGSN in Exp 1 and Exp 2 and the ratio Cy5/Cy3 (Gel C-006). The middle illustration of FIG. 4(A) shows that the Western blot method is used to detect pGSN in the ET samples and LT samples of patients Nos. 2779, 2792 and 2766 and the mixture thereof, wherein the high-concentration proteins in plasma have been removed. The bottom illustration of FIG. 4(A) shows the samples are processed with electrophoresis and stained with commassie blue. FIG. 4(B) shows the variation of pGSN concentrations of 32 pairs of primitive plasma samples (no high-concentration protein is removed) taken at ET and LT, wherein 1 μl of each sample is taken for electrophoresis processing. FIG. 4(C) shows the result of the nonparametric paired-sample analysis for the significance of pGSN concentrations in 32 pairs of ET and LT samples, wherein the horizontal lines represent the averages of pGSN concentrations.

FIG. 5 shows that the special peptide sequence of pGSN is used to fabricate a pGSN-specific polyclonal antibody. FIG. 5(A) shows the amino acid sequence of the full-length pGSN, which is translated from the full-length cDNA sequence of pGSN (GenBank accession number NM_{—}000177), wherein the boldfaced sequences are the sequences of signal peptides, and wherein the boldfaced and underlined sequence is the antigen peptide sequence used to fabricate anti-pGSN N20 antibody. In FIG. 5(B), the Western blot method is used to detect GSN and pGSN and verify the specificity of the anti-pGSN N20 antibody and the anti-GSN monoclonal antibody (BD Biosciences Clontech) after the SDS-gel electrophoresis has fractionated 40 μg of the cell extract of the CRC cell line (SW480) (CE), 40 μg of the cell culture medium (CM), 1 μl of plasma of healthy persons (PH) and 1 μl of plasma of CRC patients.

FIG. 6 shows the blood sample-based ELISA method for quantitatively determining pGSN. In FIG. 6(A), the SDS-PAGE electrophoresis is used to analyze the purified full-length pGSN protein generated by E. coli. FIG. 6(B) shows the architecture of the ELISA method for quantitatively determining pGSN specifically, wherein the anti-gelsolin N20 polyclonal antibody, the commercial anti-gelsolin antibody able to recognize two subtypes of gelsolins (BD Biosciences, San Jose, Calif., USA), and the full-length pGSN are respectively used as the primary antibody, the secondary antibody and the standard protein sample of the standard calibration curve. In FIG. 6(C), the above-mentioned ELISA technology is used to establish the standard curve for determining the full-length pGSN concentration ranging from 9.375 to 250 ng/ml.

FIG. 7 shows that the in-house-developed ELISA technology is used to detect the pGSN concentrations in the plasma samples of the CRC patients of different stages, wherein the ELISA technology is used to analyze the variation of pGSN in the blood samples of healthy agemates (the control group (n=25)) and the blood samples of 149 CRC patients of different stages (n=28-45). The analysis results are presented with a box chart, and the horizontal lines represent the average values. The nonparametric unpaired-sample analysis technology is used to analyze the significance of the statistic differences in the pGSN concentrations of different stages.

FIG. 8 shows overexpression of secretory gelsolin in CRC tissues. In FIG. 8(A), Immunohistochemical staining of secretory gelsolin with anti-gelsolin N20 antibodies in paired tumor (T) and adjacent non-tumor (AN) tissues from two representative cases (scale bar-200 μm). The boxed areas indicated in the upper panels are enlarged and shown in the lower panels. In FIG. 8(B), the anti-gelsolin N20 antibody is used to IHC-stained tissue sections of 148 CRC patients for analyzing pGSN expression in tumor tissues (T) and adjacent normal tissues (AN). The expression is scored according to expression strength (+, ++, +++) and expression ratio (0-100%). The full score is 300. The expression scoring greater than 150 is assigned to be the strong staining. The expression scoring equal to or smaller than 150 is assigned to weak staining or negative staining. FIG. 8(B) shows statistical analysis (McNemar's test) of the percentage of strong and negative/weak staining of pGSN in CRC tissue sections and normal tissue sections. In FIG. 8(C), the statistic paired t-test is used to analyze the significance of expression variation in 133 sections containing both tumor tissue and adjacent normal tissue, wherein TAN is the difference between the score of tumor (T) tissue and the score of adjacent normal (AN) tissue.

FIG. 9 shows effects of secretory gelsolin on SW480 cell migration in vitro. In FIG. 9(A), SW480 cells are seeded to the upper chamber of a Transwell assay module and either left untreated (No Ab) or incubated with anti-gelsolin antibodies or other control polyclonal antibodies (Ab-1 to Ab-4, 2 μg each). In FIG. 9(B), purified His-tagged recombinant full-length secretory gelsolin (2 μg/ml) is added to the lower chamber (Low-2) or upper chamber (2 or 5 μg/ml; Up-2 or Up-5) of a Transwell assay module containing SW480 cells. In FIG. 9(C), the experiment is set up as described in (B), except that anti-gelsolin antibodies (2 μg) are added to the upper chamber. In FIG. 9(D), upper panel—Western blot analysis of the levels of cytoplasmic and secretory gelsolin in cell extracts (CE) or conditioned media (CM) from parental SW480 cells or SW480 cells transiently transfected without (mock) or with the Myctagged-GSN-FL plasmid (pGSN-FL). Middle panel—SW480 cells transiently transfected without (mock) or with pGSN-FL were subjected to a Transwell migration assay. Representative microphotographs of filters and a quantitative analysis of the assay results from two independent experiments are shown. Lower panel—SW480 cells transiently transfected without (mock) or with pGSNFL were subjected to a wound healing assay for 48 h.

### DETAILED DESCRIPTION OF THE INVENTION

The characteristics and advantages of the present invention will be demonstrated with embodiments below. However, these embodiments are only to exemplify the present invention. The scope of the present invention would not be constrained by the embodiments, and the instruments, devices, methods, results, titles and sub-titles involved in the embodiments.

### EMBODIMENTS

### Embodiment I

### Searching for CRC Metastasis-Associated Biomarkers in Blood

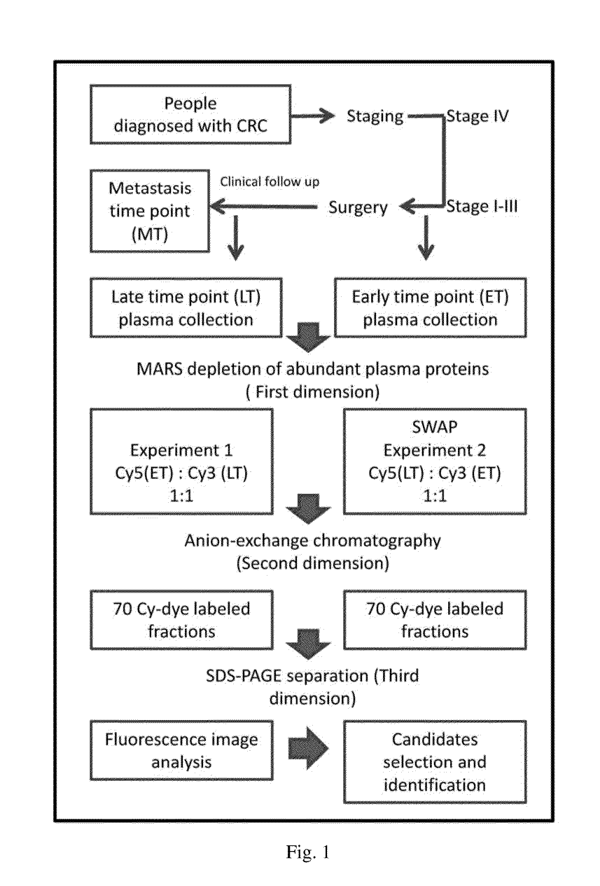

Some research reports point out the fact: Analyzing the intensity of fluorescent images with proteomics platform, the fluorescent labeling technology and the multi-dimensional fractionation system can successfully find out differentially-expressing proteins in the blood samples of the patients of nasopharyngeal cancer. These proteins are candidates for blood biomarkers of nasopharyngeal cancer (refer to Wu, C. C., et al. 2008, Proteomics 8: 3605-3620, and Peng, P. H. et al. 2011, J Proteomics 74: 744-757). The Inventors used a similar platform to search for promising CRC metastasis-associated biomarkers in the blood samples of the patients at different stages of CRC. In the embodiment, the Inventors used the proteomic technology to search for CRC metastasis-associated biomarkers in the blood samples of CRC patients, and the flowchart of the search strategy is shown in FIG. 1.

From 1995 to 2003, the Inventors collected the plasma samples of 32 patients at two different time points. The time points of collecting the samples and the clinical characteristics of the patients are shown in Table. 1. The early time point (ET) is referred to the time point that the patient was newly diagnosed to have CRC. The late time point is referred the time point of sampling nearest to the time point that the patient was diagnosed to have CRC metastasis.

TABLE 1
the time points of collecting the plasma samples and the clinical
characteristics of the patients

| | | | | TNM | | | | | |
|---|---|---|---|---|---|---|---|---|---|
| | | | | stage at | Differentiation | | ET^{a} | LT^{b} | |
| No. of | | | | initial | grading in | Metastasized | (CEA | (CEA | |
| patient | Age | Gender | Location | diagnosis | histology | organ | ng/ml) | ng/ml) | MT^{c} |
| 2766^{e} | 43 | F | colon | IIIc | well | liver | 2000-05-29(1.5) | 2000-02-9 | 2001-02-13 |
| | | | | | | | | (87.1) | |
| 2779^{e} | 63 | M | rectum | IIIa | moderate | liver | 2000-06-19(6.2) | 2001-04-10 | 2001-04-18 |
| | | | | | | | | (725.0) | |
| 2792^{e} | 66 | M | rectum | IIb | moderate | liver | 2000-07-13(41.5) | 2000-10-13 | 2000-11-01 |
| | | | | | | | | (394.8) | |
| 642 | 60 | M | rectum | IIa | moderate | lung | 1995-11-16(13.6) | 1999-08-17 | 1999-10-15 |
| | | | | | | | | (7.7) | |
| 1773 | 66 | M | rectum | Ib | poor | liver | 1996-12-30(4.6) | 2002-05-14 | 2002-05-23 |
| | | | | | | | | (14.5) | |
| 1738 | 36 | F | colon | IIIa | moderate | liver | 1997-01-06(6.5) | 2000-08-29 | 2000-08-25 |
| | | | | | | | | (37.6) | |
| 2029 | 68 | M | colon | IIa | moderate | lung | 1997-07-07(1.7) | 2000-01-05 | 2000-01-27 |
| | | | | | | | | (3.9) | |
| 2365 | 67 | M | colon | IIIb | moderate | lung | 1998-07-11(21.1) | 2003-01-09 | 2002-12-16 |
| | | | | | | | | (4.4) | |
| 2394 | 30 | F | rectum | IIIa | moderate | liver | 1998-07-30(23.9) | 2001-05-22 | 2001-07-20 |
| | | | | | | | | (16.0) | |
| 2511 | 61 | M | rectum | Ib | moderate | lung | 1998-10-22(3.8) | 2000-05-26 | 2001-05-18 |
| | | | | | | | | (12.9) | |
| 2638 | 74 | F | rectum | IIIc | poor | another | 1999-05-10(6.0) | 2000-05-20 | 2000-06-02 |
| | | | | | | organ | | (1013.0) | |
| 2698 | 79 | F | rectum | IIIb | moderate | lung | 1999-10-07(2.1) | 2001-08-07 | 2001-11-10 |
| | | | | | | | | (3.4) | |
| 2706 | 64 | M | rectum | IIa | moderate | liver | 1999-11-025(38.9) | 2001-11-06 | 2001-11-14 |
| | | | | | | | | (35.5) | |
| 2724 | 71 | F | rectum | IIIc | moderate | liver | 2000-03-02 | 2001-02-20 | 2001-03-14 |
| | | | | | | | (110.0) | (32.8) | |
| 2746 | 55 | F | rectum | IIIc | poor | lung | 2000-05-01(3.5) | 2001-12-25 | 2002-02-26 |
| | | | | | | | | (1.3) | |
| 2800 | 43 | F | colon | IIIb | well | another | 2000-07-31(10.2) | 2003-03-11 | 2003-03-28 |
| | | | | | | organ | | (197.8) | |
| 2804 | 76 | F | rectum | IIIb | moderate | lung | 2000-08-14 | 2001-08-17 | 2001-08-19 |
| | | | | | | | (184.8) | (1983.8) | |
| 2828 | 71 | F | rectum | IIIb | moderate | another | 2000-10-09(2.7) | 2002-06-04 | 2002-06-07 |
| | | | | | | organ | | (1.5) | |
| 2833 | 75 | M | colon | IIb | poor | lung | 2000-10-16(25.8) | 2001-10-30 | 2002-01-24 |
| | | | | | | | | (1621.6) | |
| 2846 | 78 | M | colon | IIIa | well | liver | 2000-11-20(3.2) | 2001-03-20 | 2001-08-04 |
| | | | | | | | | (9.2) | |
| 2890 | 74 | M | rectum | IIIa | moderate | liver | 2001-03-12(9.8) | 2002-01-24 | 2002-01-08 |
| | | | | | | | | (17.5) | |
| 2935 | 66 | F | rectum | IIIa | moderate | lung | 2001-03-22(3.7) | 2003-09-25 | 2003-10-03 |
| | | | | | | | | (12.7) | |
| 2969 | 58 | F | rectum | IIIb | moderate | lung | 2001-04-02(—) | 2002-02-05 | 2002-02-28 |
| | | | | | | | | (4.0) | |
| 2981 | 53 | M | rectum | IIIb | poor | liver | 2001-04-29(—) | 2001-08-07 | 2001-08-08 |
| | | | | | | | | (229.1) | |
| 3189 | 75 | M | colon | IIIc | moderate | another | 2001-07-25(91.0) | 2001-02-20 | 2003-07-17 |
| | | | | | | organ | | (565.7) | |
| 3282 | 74 | M | rectum | IIIc | poor | liver | 2001-09-08(1.9) | 2002-04-22 | 2002-05-28 |
| | | | | | | | | (8.8) | |
| 3347 | 76 | M | colon | IIb | well | liver | 2001-10-12(3.1) | 2002-10-03 | 2002-10-15 |
| | | | | | | | | (60.6) | |
| 3369 | 51 | M | rectum | IIIa | moderate | lung | 2001-10-24(7.9) | 2003-03-14 | 2003-03-31 |
| | | | | | | | | (5.4) | |
| 3419 | 59 | M | rectum | IIIa | moderate | another | 2001-11-13(33.4) | 2002-06-08 | 2002-05-13 |
| | | | | | | organ | | (727.4) | |
| 3503 | 69 | F | colon | IIIc | moderate | lung | 2001-12-27(26.5) | 2003-02-11 | 2003-02-18 |
| | | | | | | | | (18.6) | |
| 3530 | 62 | F | rectum | IIb | moderate | liver | 2002-01-08(16.1) | 2002-09-02 | 2002-09-30 |
| | | | | | | | | (8.7) | |
| 3581 | 74 | M | rectum | IIIa | moderate | another | 2002-01-31(3.3) | 2002-11-25 | 2003-03-31 |
| | | | | | | organ | | (5.2) | |

^{a}sample at the time point that the patient was newly diagnosed to have CRC
^{b}sample at the time point nearest to the time point that the patient was diagnosed to have CRC metastasis
^{c}the time point that the patient was diagnosed to have CRC metastasis
d: the CEA concentration is detected with a kit produced by Abbott Laboratories. Abbott Park, Illinois, USA. In ET, null CE concentration is detected in the samples of two patients (No. 2969 and No. 2981).
^{e}the samples used to search for CRC metastasis biomarkers

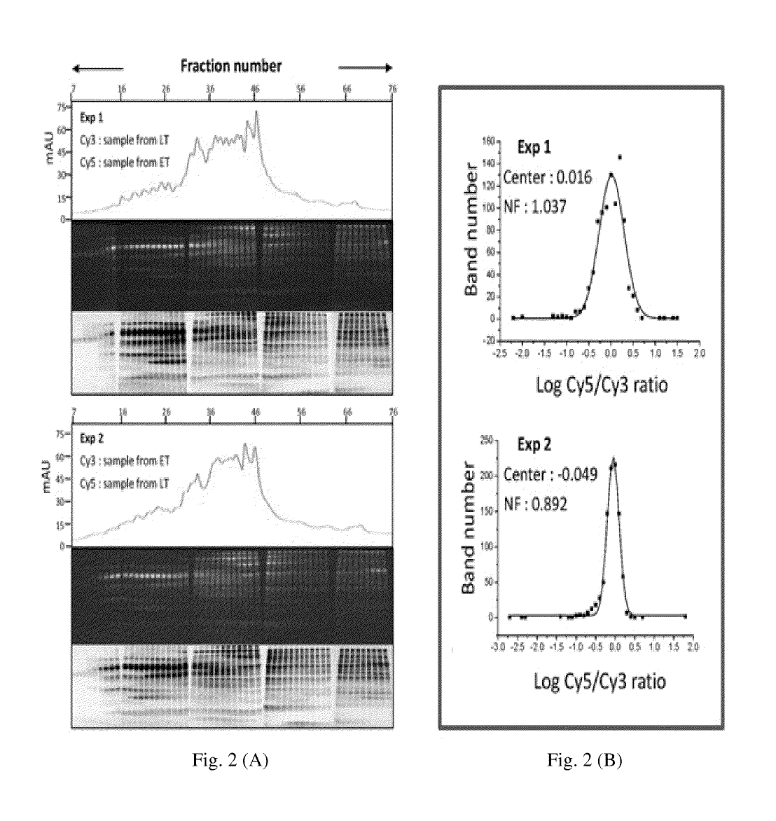

Among the 32 CRC patients, 14 patients have liver metastases; 12 patients have lung metastases; 6 patients have metastases in other organs. In order to reduce the influence of the individual difference of the samples and detect more proteins of minor concentrations, the Inventors removed 6 primary and high-concentration proteins, including albumin, IgG, antitrypsin, IgA, transferring and haptoglobin, from 3 pairs (ET and LT) of ET plasma samples and LT plasma samples of 3 patients. Then, the 3 pairs of plasma samples are mixed by a ratio of 1:1:1 to form a pair of ET plasma sample and LT plasma sample. The pair of samples is respectively fluorescence-labeled with Cy3 and Cy5 (Exp 1). The pair of samples is also respectively fluorescence-labeled with Cy5 and Cy3 (Exp 2). Next, the 2 samples are mixed by a ratio of 1:1. Next, the mixture is processed with the ion exchange resin chromatography and the SDS-PAGE electrophoresis to fractionate proteins, as shown in FIG. 2.

After the anion exchange resin chromatography has fractionated the proteins in the mixture sample, a spectrophotometer measures the absorbance of the proteins of each tube of sample at a wavelength of 280 nm, as shown in the upper illustrations of FIG. 2A. Next, use 10% SDS-gel electrophoresis to further fractionate the proteins in the tubes No. 7-No 76. Next, use the Typhoon 9400 fluorescent image analyzer to take the images of Cy3 and Cy5 from each electrophoresis gel, as shown in the middle illustrations of FIG. 2A. Next, use the image processing software ImageQuant to systematically compare the images. Next, edit and assign numbers to the band signals of each protein. Herein, the images of Cy5 are used as the exemplification. Next, use silver nitrate to dye the proteins in the electrophoresis gels. Next, use a gray-level image analyzer to take the images of the dyed proteins. Next, overlap the gray-level images on the abovementioned band signals of the proteins, as shown in the lower illustrations of FIG. 2A.

The upper diagram and lower diagram of FIG. 2B respectively show the distributions of log Cy5/Cy3 in Exp 1 and Exp 2. The solid curves are the Gaussian distribution curves describing the distributions of log Cy5/Cy3. The x axis denotes the value of log Cy5/Cy3, and the y axis denotes the occurrence frequency of log Cy5/Cy3. The peaks of the distributions of log Cy5/Cy3 in Exp 1 and Exp 2 respectively appear at 0.016 and −0.049 of the x axis, which are used to work out that the normalization factors of Exp 1 and Exp 2 are separately 1.037 and 0.892.

The Inventors use a fluorescent image analyzer to tale all the images of the fluorescence-labeled proteins in the electrophoresis gel. Next, use the software to systematically compare the images, and select the protein signals having obvious change in the fluorescence amounts in the ET sample and the LT sample. Next, identify the proteins. After being dyed with silver nitrate, the corresponding proteins are cut off from the electrophoresis gels. Next, use mass spectrometers (MALDI-TOF MS and LC-MS/MS) to identify the proteins. Next, use the Western blot method and the immunoassay to verify whether a specified protein can function as a biomarker in tests.

### Embodiment II

### Analysis of Fluorescent Images and Identification of Differentially-Expressing Proteins

In this embodiment, use image analysis software to quantitatively measure the fluorescence of each protein in the electrophoresis gels, and select the candidates of blood cancer biomarkers, whose fluorescence values vary in different groups. Using the preset fluorescence value as the screening condition, the Inventors found 15 proteins whose fluorescence increase after CRC metastasis and 15 proteins whose fluorescence decrease after CRC metastasis. Next, cut off the bands containing the 30 candidate proteins from the electrophoresis gels respectively. Next, perform enzymatic hydrolysis of the proteins in the gel bands with Trypsin, and use mass-spectrometers (MALDI-TOF MS and MicrOTOF-Q MS) to analyze the hydrolyzed candidate proteins.

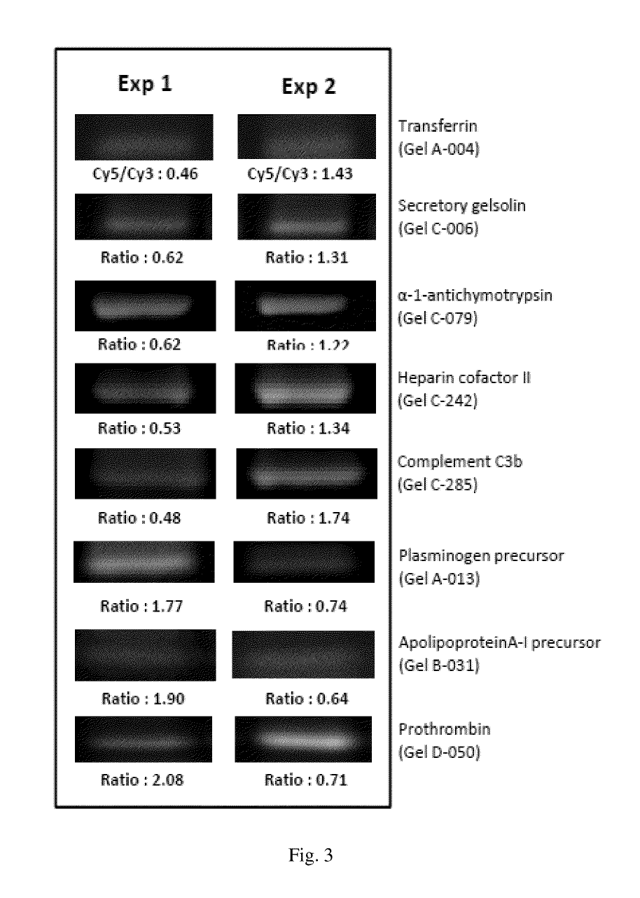

Among the 30 gel bands, the Inventor found 5 proteins whose concentrations increase after distal metastasis, including serotransferrin, pGSN, alpha-1-antichymotrypsin, heparin cofactor 2 and Complement C3b; the Inventors also found 3 proteins whose concentration decrease after distal metastasis, including plasminogen, thrombogen, and apolipoprotein A1. The details of the analysis are shown in Table. 2. The fluorescent images of the differentially-expressing proteins are shown in FIG. 3. The identification data of the CRC metastasis-associated proteins include the peptide mass fingerprints by MALDI-TOF and the corresponding sequences, and the MS/MS mass spectrograms of each molecule containing two segments of peptides by MicrOTOF-Q MS.

TABLE 2
Potential CRC metastasis-associated proteins identified by mass
spectrometry

| | | | | | | Score^{c} (% Seq |
|---|---|---|---|---|---|---|
| | | Accession | Ratio | | Expect | Cov^{d}/No. of |
| Band No.^{a} | Protein name | No.^{b} | Exp1 | Exp2 | Value | masses matched) |

Proteins with increased levels at LT

| | | | | | | |
|---|---|---|---|---|---|---|
| Gel A-004 | Serotransferrin precursor | P02787 | 0.46 | 1.43 | 5.1e−011 | 146 (31/27) |
| Gel A 020 | Serotransferrin precursor | P02787 | 0.56 | 1.30 | 6.3e−009 | 124 (22/17) |
| Gel C-006 | Secretory gelsolin precursor | P06396 | 0.62 | 1.31 | 1.3e−008 | 122 (25/19) |
| Gel C-056 | Secretory gelsolin precursor | P06396 | 0.43 | 1.83 | 0.0011 | 72 (15/12) |
| Gel C-079 | α-1-antichymotrypsin | P01011 | 0.62 | 1.22 | 1.2e−005 | 92 (33/13) |
| Gel C-242 | Heparin cofactor 2 | P05546 | 0.53 | 1.34 | 0.00023 | 78 (32/20) |
| Gel C-285 | Complement C3b | P01024 | 0.48 | 1.74 | 4.1e−012 | 157 (19/33) |

Proteins with decreased levels at LT

| | | | | | | |
|---|---|---|---|---|---|---|
| Gel A-013 | Plasminogen precursor | P00747 | 1.77 | 0.74 | 0.00051 | 76 (19/16) |
| Gel A-019 | Plasminogen precursor | P00747 | 1.86 | 0.79 | 0.0015 | 70 (14/13) |
| Gel A-044 | Plasminogen precursor | P00747 | 1.45 | 0.69 | 2.3e−008 | 128 (15/12) |
| Gel B-031 | Apolipoprotein A1 precursor | P02647 | 1.90 | 0.64 | 7.9e−012 | 153 (45/14) |
| Gel B-047 | Apolipoprotein A1 precursor | P02647 | 2.15 | 0.70 | 6.7e−006 | 94 (36/10) |
| Gel B-065 | Apolipoprotein A1 precursor | P02647 | 1.98 | 0.77 | 0.018 | 59 (31/18) |
| Gel D-050 | Prothrombin | P19221 | 2.08 | 0.71 | 2e−008 | 120 (27/14) |
| Gel D-117 | Prothrombin | P19221 | 1.45 | 0.31 | 1e−05 | 113 (17/9) |

^{a}Numbering of the protein bands
^{b}Swiss-Prot accession numbers of identified proteins
^{c}Mascot scores of identified proteins by peptide mass fingerprints
^{d}Percent sequence coverage (% Seq Cov) of matched peptides in the identified proteins

### Embodiment III

### Using the Western Blot Method to Prove that pGSN Increases with the Distal Metastasis of CRC

The Inventors used the Western blot method to verify whether a protein molecule pGSN is associated with the distal metastasis of CRC. In the embodiment, the pGSN protein has the following amino acid sequence (SEQ ID NO:1):

| | |
|---|---|
| 1 | Met-Ala-Pro-His-Arg-Pro-Ala-Pro-Ala-Leu-Leu-Cys-Ala-Leu-Ser-Leu-Ala-Leu-Cys-Ala- |
| 21 | Leu-Ser-Leu-Pro-Val-Arg-Ala-Ala-Thr-Ala-Ser-Arg-Gly-Ala-Ser-Gln-Ala-Gly-Ala-Pro- |
| 41 | Gln-Gly-Arg-Val-Pro-Glu-Ala-Arg-Pro-Asn-Ser-Met-Val-Val-Glu-His-Pro-Glu-Phe-Leu- |
| 61 | Lys-Ala-Gly-Lys-Glu-Pro-Gly-Leu-Gln-Ile-Trp-Arg-Val-Glu-Lys-Phe-Asp-Leu-Val-Pro- |
| 81 | Val-Pro-Thr-Asn-Leu-Tyr-Gly-Asp-Phe-Phe-Thr-Gly-Asp-Ala-Tyr-Val-Ile-Leu-Lys-Thr- |
| 101 | Val-Gln-Leu-Arg-Asn-Gly-Asn-Leu-Gln-Tyr-Asp-Leu-His-Tyr-Trp-Leu-Gly-Asn-Glu-Cys- |
| 121 | Ser-Gln-Asp-Glu-Ser-Gly-Ala-Ala-Ala-Ile-Phe-Thr-Val-Gln-Leu-Asp-Asp-Tyr-Leu-Asn- |
| 141 | Gly-Arg-Ala-Val-Gln-His-Arg-Glu-Val-Gln-Gly-Phe-Glu-Ser-Ala-Thr-Phe-Leu-Gly-Tyr- |
| 161 | Phe-Lys-Ser-Gly-Leu-Lys-Tyr-Lys-Lys-Gly-Gly-Val-Ala-Ser-Gly-Phe-Lys-His-Val-Val- |
| 181 | Pro-Asn-Glu-Val-Val-Val-Gln-Arg-Leu-Phe-Gln-Val-Lys-Gly-Arg-Arg-Val-Val-Arg-Ala- |
| 201 | Thr-Glu-Val-Pro-Val-Ser-Trp-Glu-Ser-Phe-Asn-Asn-Gly-Asp-Cys-Phe-Ile-Leu-Asp-Leu- |
| 221 | Gly-Asn-Asn-Ile-His-Gln-Trp-Cys-Gly-Ser-Asn-Ser-Asn-Arg-Tyr-Glu-Arg-Leu-Lys-Ala- |
| 241 | Thr-Gln-Val-Ser-Lys-Gly-Ile-Arg-Asp-Asn-Glu-Arg-Ser-Gly-Arg-Ala-Arg-Val-His-Val- |
| 261 | Ser-Glu-Glu-Gly-Thr-Glu-Pro-Glu-Ala-Met-Leu-Gln-Val-Leu-Gly-Pro-Lys-Pro-Ala-Leu- |
| 281 | Pro-Ala-Gly-Thr-Glu-Asp-Thr-Ala-Lys-Glu-Asp-Ala-Ala-Asn-Arg-Lys-Leu-Ala-Lys-Leu- |
| 301 | Tyr-Lys-Val-Ser-Asn-Gly-Ala-Gly-Thr-Met-Ser-Val-Ser-Leu-Val-Ala-Asp-Glu-Asn-Pro- |
| 321 | Phe-Ala-Gln-Gly-Ala-Leu-Lys-Ser-Glu-Asp-Cys-Phe-Ile-Leu-Asp-His-Gly-Lys-Asp-Gly- |
| 341 | Lys-Ile-Phe-Val-Trp-Lys-Gly-Lys-Gln-Ala-Asn-Thr-Glu-Glu-Arg-Lys-Ala-Ala-Leu-Lys- |
| 361 | Thr-Ala-Ser-Asp-Phe-Ile-Thr-Lys-Met-Asp-Tyr-Pro-Lys-Gln-Thr-Gln-Val-Ser-Val-Leu- |
| 381 | Pro-Glu-Gly-Gly-Glu-Thr-Pro-Leu-Phe-Lys-Gln-Phe-Phe-Lys-Asn-Trp-Arg-Asp-Pro-Asp- |
| 401 | Gln-Thr-Asp-Gly-Leu-Gly-Leu-Ser-Tyr-Leu-Ser-Ser-His-Ile-Ala-Asn-Val-Glu-Arg-Val- |
| 421 | Pro-Phe-Asp-Ala-Ala-Thr-Leu-His-Thr-Ser-Thr-Ala-Met-Ala-Ala-Gln-His-Gly-Met-Asp- |
| 441 | Asp-Asp-Gly-Thr-Gly-Gln-Lys-Gln-Ile-Trp-Arg-Ile-Glu-Gly-Ser-Asn-Lys-Val-Pro-Val- |
| 461 | Asp-Pro-Ala-Thr-Tyr-Gly-Gln-Phe-Tyr-Gly-Gly-Asp-Ser-Tyr-Ile-Ile-Leu-Tyr-Asn-Tyr- |
| 481 | Arg-His-Gly-Gly-Arg-Gln-Gly-Gln-Ile-Ile-Tyr-Asn-Trp-Gln-Gly-Ala-Gln-Ser-Thr-Gln- |
| 501 | Asp-Glu-Val-Ala-Ala-Ser-Ala-Ile-Leu-Thr-Ala-Gln-Leu-Asp-Glu-Glu-Leu-Gly-Gly-Thr- |
| 521 | Pro-Val-Gln-Ser-Arg-Val-Val-Gln-Gly-Lys-Glu-Pro-Ala-His-Leu-Met-Ser-Leu-Phe-Gly- |
| 541 | Gly-Lys-Pro-Met-Ile-Ile-Tyr-Lys-Gly-Gly-Thr-Ser-Arg-Glu-Gly-Gly-Gln-Thr-Ala-Pro- |
| 561 | Ala-Ser-Thr-Arg-Leu-Phe-Gln-Val-Arg-Ala-Asn-Ser-Ala-Gly-Ala-Thr-Arg-Ala-Val-Glu- |
| 581 | Val-Leu-Pro-Lys-Ala-Gly-Ala-Leu-Asn-Ser-Asn-Asp-Ala-Phe-Val-Leu-Lys-Thr-Pro-Ser- |
| 601 | Ala-Ala-Tyr-Leu-Trp-Val-Gly-Thr-Gly-Ala-Ser-Glu-Ala-Glu-Lys-Thr-Gly-Ala-Gln-Glu- |
| 621 | Leu-Leu-Arg-Val-Leu-Arg-Ala-Gln-Pro-Val-Gln-Val-Ala-Glu-Gly-Ser-Glu-Pro-Asp-Gly- |
| 641 | Phe-Trp-Glu-Ala-Leu-Gly-Gly-Lys-Ala-Ala-Tyr-Arg-Thr-Ser-Pro-Arg-Leu-Lys-Asp-Lys- |
| 661 | Lys-Met-Asp-Ala-His-Pro-Pro-Arg-Leu-Phe-Ala-Cys-Ser-Asn-Lys-Ile-Gly-Arg-Phe-Val- |
| 681 | Ile-Glu-Glu-Val-Pro-Gly-Glu-Leu-Met-Gln-Glu-Asp-Leu-Ala-Thr-Asp-Asp-Val-Met-Leu- |
| 701 | Leu-Asp-Thr-Trp-Asp-Gln-Val-Phe-Val-Trp-Val-Gly-Lys-Asp-Ser-Gln-Glu-Glu-Glu-Lys- |
| 721 | Thr-Glu-Ala-Leu-Thr-Ser-Ala-Lys-Arg-Tyr-Ile-Glu-Thr-Asp-Pro-Ala-Asn-Arg-Asp-Arg- |
| 741 | Arg-Thr-Pro-Ile-Thr-Val-Val-Lys-Gln-Gly-Phe-Glu-Pro-Pro-Ser-Phe-Val-Gly-Trp-Phe- |
| 761 | Leu-Gly-Trp-Asp-Asp-Asp-Tyr-Trp-Ser-Val-Asp-Pro-Leu-Asp-Arg-Ala-Met-Ala-Glu-Leu- |
| 781 | Ala-Ala |

It should be understood by the persons skilled in the art: the amino acids in SEQ ID NO:1 can be replaced to form different sequences with similar features. Therefore, the present invention does not constrain that the CRC metastasis-associated biomarker pGSN should be only in form of SEQ ID NO:1. In other embodiments, the pGSN proteins 95% resembling SEQ ID NO:1 also function as CRC metastasis-associated biomarkers.

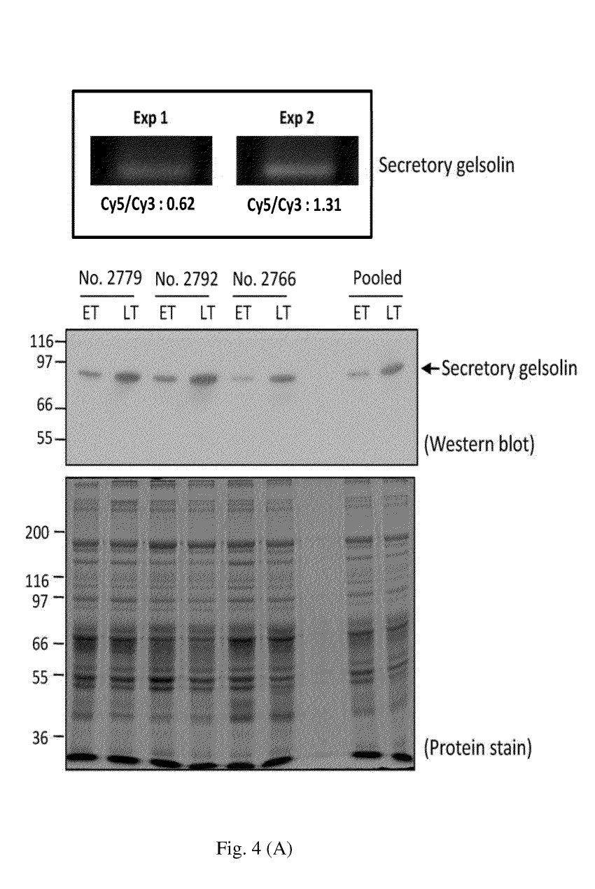

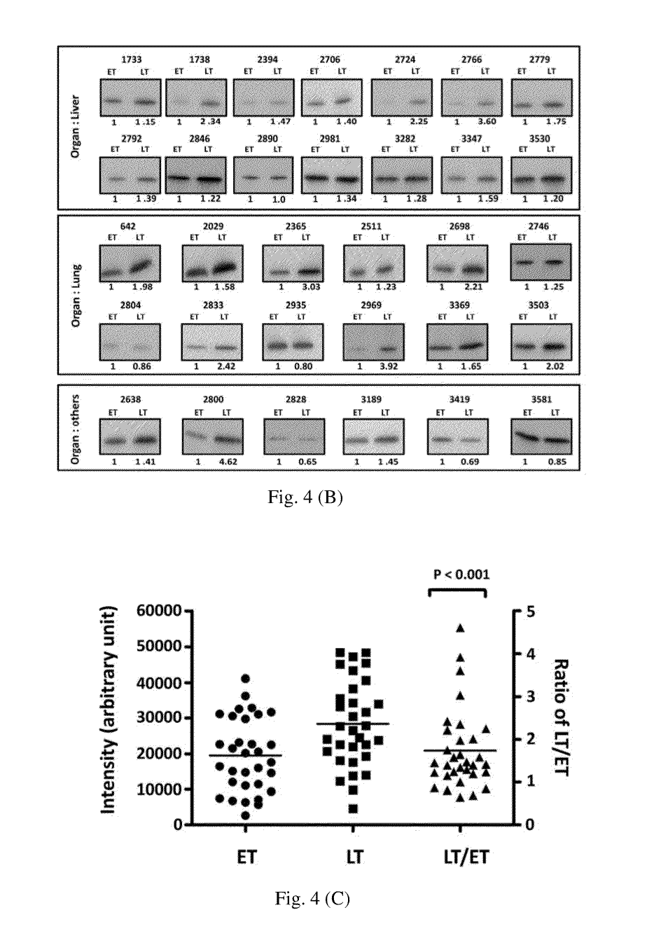

In FIG. 4A, the fluorescent images of pGSN in the electrophoresis gels imply that the pGSN protein in the blood samples of the patient has significant variation. The family of human pGSN includes two subtype members: cytoplasmic gelsolin and secretory gelsolin (also called plasma gelsolin, and abbreviated as pGSN). Compared with the cytoplasmic gelsolin, pGSN has an additional amino acid sequence containing 25 amino acids in the N-tei urinal thereof (Refer to Pei, H. et al. 2007, J Proteome Res 6: 2495-2501). There is an anti-gelsolin antibody produced by BD Biosciences, San Jose, Calif., USA and available in the market. The anti-gelsolin antibody can recognize two subtypes of gelsolins. The Inventor perform Western blot tests on 3 pairs of plasma samples of 3 CRC patients with the anti-gelsolin antibody. The results show that the concentration of pGSN increases in the plasma samples obtained at LT, as shown in FIG. 4A.

Next, the Inventors take a 1 μl sample from each of the primitive plasma samples (the plasma samples where the high-concentration proteins are preserved). Next, use the Western blot method to evaluate the concentration variation of pGSN at ET and LT. The results show that the Western blot method can detect pGSN in a tiny quantity (1 μl) of plasma sample. The results also show that pGSN of 26 pairs of plasma samples increases at LT among 32 pairs of plasma samples of CRC patients, as shown in FIG. 4B. The result of statistic analysis shows that the LT pGSN concentration is 1.67(±0.84) times higher than the ET pGSN concentration for CRC patients. Among 32 pairs of plasma samples, the group of liver metastasis has 14 pairs of plasma samples, and pGSN increases in 13 pairs thereof; the group of lung metastasis has 12 pairs of plasma sample, and pGSN increases in 10 pairs thereof; the group of metastases to other organs has 6 pairs of plasma samples, and pGSN increases in 3 pairs thereof. The ratios of pGSN-increasing samples are respectively 93%, 83% and 50% in the corresponding groups, as shown in FIG. 4C.

### Embodiment IV

### Fabricating pGSN-Specific Polyclonal Antibodies

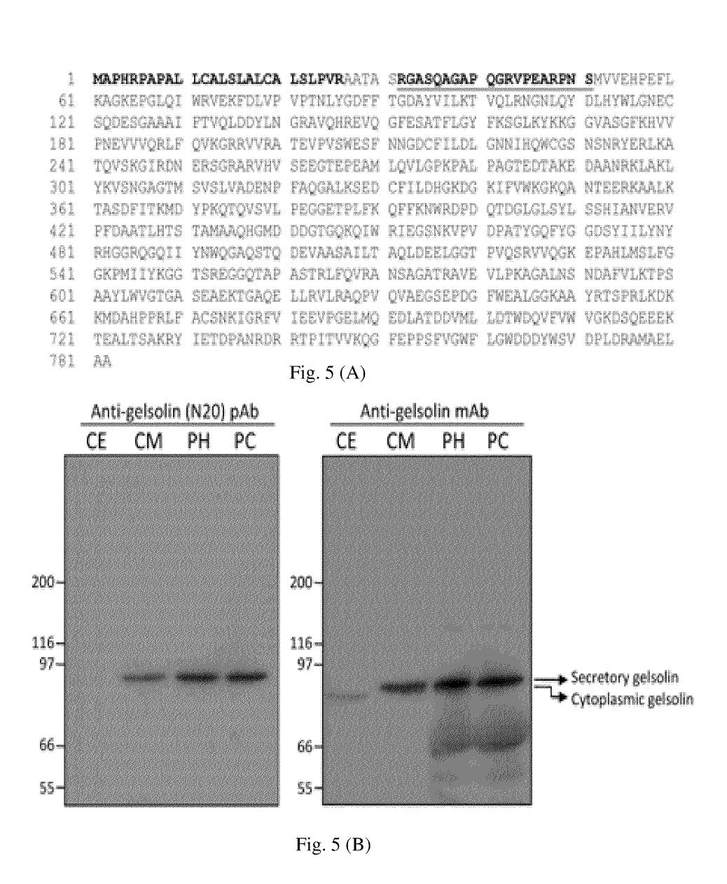

Most of the anti-gelsolin antibodies available in the market cannot distinguish two subtypes of gelsolins. This fact indicates that there is not yet any pGSN-specific antibody so far. There is a special sequence of 20 amino acids: RGASQAGAPQGRVPEARPNS (SEQ ID NO:2) existing in the N-terminal of pGSN but not existing in cytoplasmic gelsolin. The special sequence is called the N20 peptide, which is located in the residue 32-51, as shown in FIG. 5A. The peptide and BSA (Bovine Serum Albumin) are coupled by glutaraldehyde to function as an antigen. The antigen is injected into New Zealand white rabbits to induce an immunological reaction. Next, collect the serum. The in-house-developed affinity column containing the N20 peptide is used to purify an N20-peptide-specific polyclonal antibody (called the anti-gelsolin N20 antibody thereinafter) from the serum.

Via the Western blot tests, it is proved that the anti-gelsolin antibodies available in the market, which are fabricated with the carbon-terminal amino acid sequence simultaneously existing in the two subtypes of gelsolins being the antigen, can recognize each or both of the two subtypes of gelsolins (cytoplasmic gelsolin and plasma gelsolin) in cell extract and concentrated cell cultivation liquid. Contrarily, the anti-gelsolin N20 polyclonal antibody would not recognize the non-secretory cytoplasmic gelsolin but can only recognizes pGSN in cell cultivation liquid, as shown in FIG. 5B. Thus is proved that the in-house-developed anti-gelsolin N20 polyclonal antibody can specifically recognize pGSN. Besides, the Inventors also use the Western blot method to analyze the plasma samples of CRC patients and healthy persons. The experimental results show that pGSN is the primary subtype of gelsolin existing in the human plasma samples.

### Embodiment V

### Establishing ELISA Method Able to Quantitatively Determine pGSN in Blood Samples

Firstly, use a specified primer pair, including a forward primer: 5′-GGATCCCCATGGCTCCGCACCGCCCC-3′ (SEQ ID NO:3), and a reverse primer: 5′-AAGCTTTCAGGCAGCCAGCTCAGCCAT-3′ (SEQ ID NO:4), to undertake PCR (Polymerase Chain Reaction) to amplify the full-length pGSN cDNA from the cDNA template of the CRC cell line SW480. Next, use the gene editing technology to divide the amplified full-length pGSN cDNA into segments and connect the segments to the pGEM-T vector (Promega, Madison, Wis., USA), whereby to form an expression plasmid pGEM-T/GSN-FL. Next, the expression plasmid is sent into the E. coli host to express the full-length pGSN.

In order to establish a pGSN-specific ELISA method, the Inventors respectively use the anti-gelsolin N20 polyclonal antibody, the commercial anti-gelsolin antibody (BD Biosciences, San Jose, Calif., USA), and the full-length pGSN as the primary antibody, the secondary antibody and the standard protein sample of the standard calibration curve. The abovementioned commercial anti-gelsolin antibody can recognize two subtypes of gelsolins. The ELISA method implemented by the abovementioned combination can detect the full-length pGSN having a concentration ranging from 9.375-250 ng/ml in standard samples, as shown in FIG. 5.

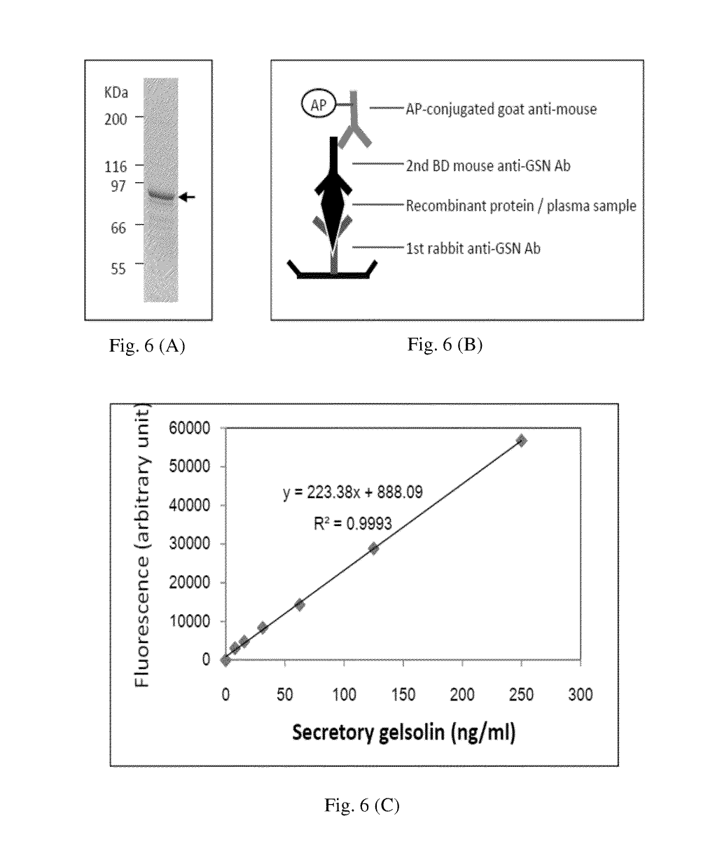

Next, use the ELISA method established above to determine the concentrations of pGSN of the 32 pairs of ET and LT plasma samples of the CRC patients. As shown in FIG. 6A, the pGSN concentrations of the LT plasmas samples (3.86±2.28 g/ml) are obviously higher than those of the ET plasmas samples (2.38±1.61 g/ml) (p<0.001, Wilcoxon signed-rank test). Similar to the previous reports, it is also found in our statistic analysis for 30 pairs of ET and LT plasma samples of the CRC patients: the CEA concentrations of the LT plasmas samples (254.5±499.5 ng/ml) are obviously higher than those of the ET plasmas samples (23.6±39.6 ng/ml) (p=0.008, Wilcoxon signed-rank test), as shown in FIG. 6B.

It is found in a further analysis: the variations of CEA concentrations of the 30 pairs of the ET and LT plasma samples are inconsistent. Post-metastasis CEA concentrations do not increase in 10 pairs of samples among the 30 pairs of ET and LT plasma samples. It is interesting and meaningful: the Inventors found that post-metastasis pGSN concentrations obviously increase in 9 pairs of samples among the pairs of samples.

Refer to Table. 3. Among the 32 pairs of samples, 27 LT plasma samples were collected 1-137 days earlier before the patients were diagnosed to have metastases, and only 5 LT plasma samples (of the patients Nos. 1738, 2365, 2511, 2890 and 3419) were collected respectively 4, 24, 8, 16 and 26 days later after the patients were diagnosed to have metastases. Via analyzing the information of the samples and the pGSN concentrations, the Inventor found that the pGSN concentration of the LT sample is higher than that of the ET sample in 22 of the 27 pairs of samples, which is verified with the Western blot method and the ELISA method.

TABLE 3
Results of Western blot test and ELISA measurement for 32 pairs
of plasma samples

| | | ET^{a} | LT^{b} | | | pGSN ratio | |
|---|---|---|---|---|---|---|---|
| No. of | Metastasized | (CEA ng/ml/ | (CEA ng/ml/ | | MT − LT | (LT/ET >1, times) | |
| patient | organ | pGSNμg/ml) | pGSN μg/ml) | MT^{c} | (day) | WB | ELISA |
| 2766^{d} | liver | 2000-05-29 (1.5/1.30) | 2001-02-09 (87.1/3.50) | 2001-02-13 | 4 | yes, 3.60 | yes, 2.69 |
| 2779^{d} | liver | 2000-06-19 (6.2/2.99) | 2001-04-10 (725.0/4.14) | 2001-04-18 | 8 | yes, 1.75 | yes, 1.38 |
| 2792^{d} | liver | 2000-07-13 (41.5/2.01) | 2000-10-13 (394.8/4.07) | 2000-11-01 | 19 | yes, 1.39 | yes, 2.02 |
| 642 | Lung | 1995-11-16 (13.6/0.82) | 1999-08-17 (7.7/0.57)^{e} | 1999-10-15 | 59 | yes, 1.98^{f} | no, 0.70^{f} |
| 1773 | liver | 1996-12-30 (4.6/3.60) | 2002-05-14 (14.5/4.38) | 2002-05-23 | 9 | yes, 1.15 | yes, 1.22 |
| 1738 | liver | 1997-01-06 (6.5/2.70) | 2000-08-29 (37.6/3.30) | 2000-08-25 | −4 | yes, 2.34 | yes, 1.22 |
| 2029 | Lung | 1997-07-07 (1.7/4.02) | 2000-01-05 (3.9/4.55) | 2000-01-27 | 22 | yes, 1.58 | yes, 1.13 |
| 2365 | Lung | 1998-07-11 (21.1/0.82) | 2003-01-09 (4.4/1.96)^{e} | 2002-12-16 | −24 | yes, 3.03 | yes, 2.39 |
| 2394 | liver | 1998-07-30 (23.9/4.60) | 2001-05-22 (16.0/8.21)^{e} | 2001-07-20 | 59 | yes, 1.47 | yes, 1.78 |
| 2511 | Lung | 1998-10-22 (3.8/2.04) | 2000-05-26 (12.9/4.01) | 2001-05-18 | −8 | yes, 1.23 | yes, 1.97 |
| 2638 | another | 1999-05-10 (6.0/1.25) | 2000-05-20 (1013.0/1.43) | 2000-06-02 | 13 | yes, 1.41 | yes, 1.14 |
| 2698 | Lung | 1999-10-07 (2.1/1.69) | 2001-08-07 (3.4/2.67) | 2001-11-10 | 95 | yes, 2.21 | yes, 1.58 |
| 2706 | liver | 1999-11-25 (38.9/4.02) | 2001-11-06 (35.5/4.83)^{e} | 2001-11-14 | 8 | yes, 1.40 | yes, 1.20 |
| 2724 | liver | 2000-03-02 (110.0/0.63) | 2001-02-20 (32.8/2.59)^{e} | 2001-03-14 | 22 | yes, 2.25 | yes, 4.11 |
| 2746 | Lung | 2000-05-01 (3.5/1.27) | 2001-12-25 (1.3/5.07)^{e} | 2002-02-26 | 63 | yes, 1.25 | yes, 3.99 |
| 2800 | another | 2000-07-31 (10.2/1.15) | 2003-03-11 (197.8/1.53) | 2003-03-28 | 17 | yes, 4.62 | yes, 1.33 |
| 2804 | Lung | 2000-08-14 (184.8/3.95) | 2001-08-17 (1983.8/5.43) | 2001-08-19 | 2 | no, 0.86^{f} | yes, 1.37^{f} |
| 2828 | another | 2000-10-09 (2.7/3.30) | 2002-06-04 (1.5/9.86)^{e} | 2002-06-07 | 3 | no, 0.65^{f} | yes, 2.99^{f} |
| 2833 | Lung | 2000-10-16 (25.8/1.84) | 2001-10-30 (1621.6/4.65) | 2002-01-24 | 86 | yes, 2.42 | yes, 2.53 |
| 2846 | liver | 2000-11-20 (3.2/1.64) | 2001-03-20 (9.2/3.40) | 2001-08-04 | 137 | yes, 1.22 | yes, 2.07 |
| 2890 | liver | 2001-03-12 (9.8/2.25) | 2002-01-24 (17.5/2.38) | 2002-01-08 | −16 | no, 1.00^{f} | yes, 1.06^{f} |
| 2935 | Lung | 2001-03-22 (3.7/2.05) | 2003-09-25 (12.7/2.83) | 2003-10-03 | 8 | no, 0.80^{f} | yes, 1.38^{f} |
| 2969 | Lung | 2001-04-02 (—/0.17) | 2002-02-05 (4.0/1.80) | 2002-02-28 | 23 | yes, 3.92 | yes, 10.59 |
| 2981 | liver | 2001-04-09 (—/2.87) | 2001-08-07 (229.1/3.20) | 2001-08-08 | 1 | yes, 1.34 | yes, 1.11 |
| 3189 | another | 2001-07-25 (91.0/1.50) | 2003-07-10 (565.7/2.80) | 2003-07-17 | 7 | yes, 1.45 | yes, 1.87 |
| 3282 | liver | 2001-09-08 (1.9/2.59) | 2002-04-22 (8.8/3.60) | 2002-05-28 | 36 | yes, 1.28 | yes, 1.39 |
| 3347 | liver | 2001-10-12 (3.1/1.80) | 2002-10-03 (60.6/2.47) | 2002-10-15 | 12 | yes, 1.59 | yes, 1.37 |
| 3369 | Lung | 2001-10-24 (7.9/2.02) | 2003-03-14 (5.4/3.33)^{e} | 2003-03-31 | 17 | yes, 1.65 | yes, 1.65 |
| 3419 | another | 2001-11-13 (33.4/5.24) | 2002-06-08 (727.4/4.94) | 2002-05-13 | −26 | no, 0.69 | no, 0.94 |
| 3503 | Lung | 2001-12-27(26.5/0.85) | 2003-02-11 (18.6/1.58)^{e} | 2003-02-18 | 7 | yes, 2.02 | yes, 1.86 |
| 3530 | liver | 2002-01-08 (16.1/7.40) | 2002-09-02 (8.7/11.06)^{e} | 2002-09-30 | 28 | yes, 1.20 | yes, 1.49 |
| 3581 | another | 2002-01-31 (3.3/3.88) | 2002-11-25 (5.2/3.42) | 2003-03-31 | 126 | no, 0.85 | no, 0.88 |

^{a}sample at the time point that the patient was newly diagnosed to have CRC
^{b}sample at the time point nearest to the time point that the patient was diagnosed to have CRC metastasis
^{c}the time point that the patient was diagnosed to have CRC metastasis
^{d}the sample used to search for CRC metastasis biomarkers
^{e}the CEA concentration of the LT plasma sample is not higher than that of the ET plasma sample
^{f}pGSN concentration by WT is inconsistent with pGSN concentration by ELISA

In summary, it is the Inventors that propose pGSN to function as a CRC metastasis-associated plasma biomarker for the first time in the world. Further, the Inventors also prove that the combination of pGSN and CEA can promote the sensitivity and reliability of CRC metastasis detection.

### Embodiment VI

### Using ELISA to Determine the pGSN Concentrations of Different-Stage CRC Patients

In the abovementioned experiments, the Inventors had found that post-metastasis pGSN concentration is significantly higher than pre-metastasis pGSN concentration. The statements thereinafter would address to verifying whether the plasma samples of CRC patients in different stages have different pGSN concentrations and whether pGSN concentration is associated with CRC staging. Thus are collected 25 plasma samples of healthy persons of appropriate ages and 149 plasma samples of CRC patients at different stages, including 29 at Stage I, 45 at Stage II, 37 at Stage III, and 38 at Stage IV. Then, use the in-house-developed ELISA to determine pGSN concentrations.

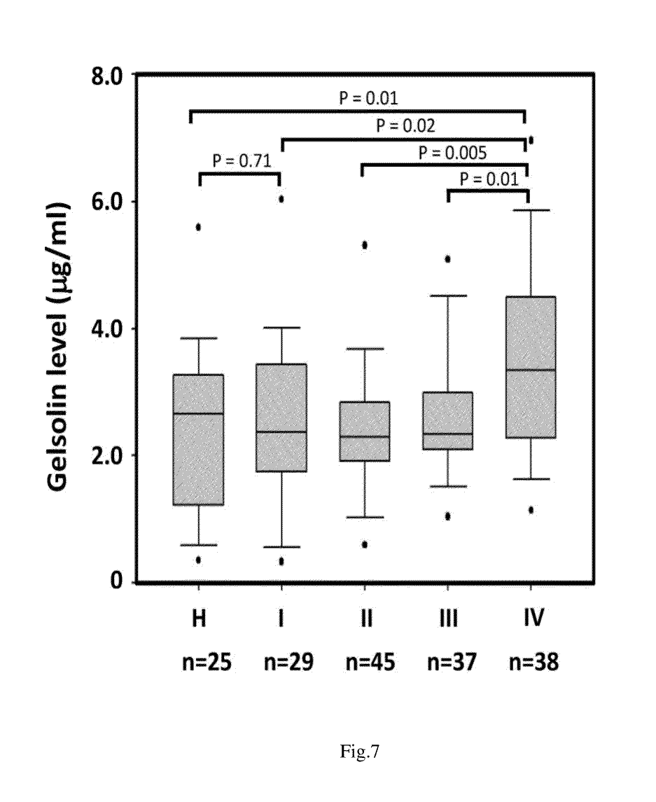

The experimental results are shown in FIG. 7. The pGSN concentrations of the patients at Stage IV are obviously higher than those of the healthy persons and the patients at Stages I-III. The pGSN concentrations of healthy persons and the patients at Stages I-IV are respectively 2.42±1.39 μg/ml (p=0.02), 2.56±1.44 μg/ml (p=0.02), 2.42±1.14 μg/ml (p=0.005), 2.60±1.06 μg/ml (p=0.01), and 3.80±2.71 μg/ml (p=0.01). Among the abovementioned 149 plasma samples, 134 plasma samples have clinical CEA data. The clinicopathologic characteristics of the 134 plasma samples are listed in Table. 4. As shown in Table. 4, pGSN concentration has obvious correlation with lymphatic metastasis and distal metastasis of CRC but lacks sufficient correlation with gender, age, cancer site, and clinicopathologic characteristics.

TABLE 4
relationship between pGSN or CEA level and clinicopathologic characteristics of the
134 CRC patients

| | | | | | |
|---|---|---|---|---|---|
| | | pGSN | | CEA | |
| | | concentration | | concentration | |
| | | (mean ± SD) | | (mean ± SD) | |
| Characteristics | No. | (□g/ml) | p value | (ng/ml) | p value |

Gender

| | | | | | |
|---|---|---|---|---|---|
| Male | 71 | 2.76 ± 1.18 | 0.061^{a} | 12.49 ± 25.17 | 0.138^{a} |
| Female | 63 | 3.38 ± 2.35 | | 192.68 ± 950.27 | |

Age (year)

| | | | | | |
|---|---|---|---|---|---|
| <65 | 67 | 2.97 ± 1.35 | 0.609^{a} | 149.99 ± 914.67 | 0.355^{a} |
| ≧65 | 67 | 3.14 ± 2.25 | | 44.43 ± 151.98 | |

Location

| | | | | | |
|---|---|---|---|---|---|
| Colon | 59 | 3.34 ± 2.33 | 0.418^{b} | 192.54 ± 984.88 | 0.281^{b} |
| Rectum | 69 | 2.84 ± 1.32 | | 24.79 ± 121.61 | |
| Others | 6 | 2.93 ± 1.93 | | 21.19 ± 23.61 | |

Histological grade

| | | | | | |
|---|---|---|---|---|---|
| Well differentiation | 24 | 2.77 ± 1.49 | 0.405^{b} | 8.83 ± 12.44 | 0.584^{b} |
| Moderate differentiation | 98 | 3.10 ± 1.95 | | 129.75 ± 764.7 | |
| Poor differentiation | 11 | 3.29 ± 1.75 | | 8.68 ± 14.46 | |
| Unclassified | 1 | 3.39 | | 3.4 | |

Histological classification

| | | | | | |
|---|---|---|---|---|---|
| Adenocarcinoma | 119 | 3.03 ± 1.87 | 0.30^{a} | 102.58 ± 693.07 | 0.26^{a} |
| Mucinous carcinoma | 12 | 3.41 ± 1.90 | | 67.85 ± 188.85 | |
| Signet ring cell adenocarcinoma | 1 | 2.18 | | 1.0 | |
| Undifferentiated carcinoma | 2 | 2.87 ± 0.73 | | 2.10 ± 1.84 | |

TNM stage

| | | | | | |
|---|---|---|---|---|---|
| Stage I | 24 | 2.50 ± 1.27 | 0.025^{b} | 3.32 ± 2.27 | <0.001^{b} |
| Stage II | 40 | 2.81 ± 1.27 | | 11.80 ± 27.50 | |
| Stage III | 36 | 2.97 ± 1.06 | | 9.56 ± 16.60 | |
| Stage IV | 33 | 3.86 ± 2.88 | | 367.56 ± 1297.53 | |
| Unknown | 1 | 2.36 | | 0.8 | |

Tumor stage

| | | | | | |
|---|---|---|---|---|---|
| Early stage (Stage I-II) | 64 | 2.70 ± 1.37 | 0.013^{a} | 8.62 ± 22.07 | 0.003^{a} |
| Late stage (Stage III-IV) | 69 | 3.40 ± 2.17 | | 180.78 ± 908.22 | |
| Unknown | 1 | 2.36 | | 0.8 | |

Lymph node metastasis (TNM-N)

| | | | | | |
|---|---|---|---|---|---|
| Negative | 72 | 2.94 ± 2.23 | 0.046^{a} | 11.90 ± 29.65 | 0.013^{a} |
| Positive | 61 | 3.21 ± 1.29 | | 199.48 ± 965.07 | |
| Unknown | 1 | 2.36 | | 0.8 | |

Tumor metastasis (TNM-M)

| | | | | | |
|---|---|---|---|---|---|
| No metastasis | 104 | 2.83 ± 1.28 | 0.002^{a} | 8.92 ± 19.86 | <0.001^{a} |
| Distal metastasis | 29 | 3.89 ± 3.04 | | 417.16 ± 1379.51 | |
| Unknown | 1 | 2.36 | | 0.8 | |

^{a}The p value is determined by Mann-Whitney U test
^{b}The p value is determined by Kruskal-Wallis H test

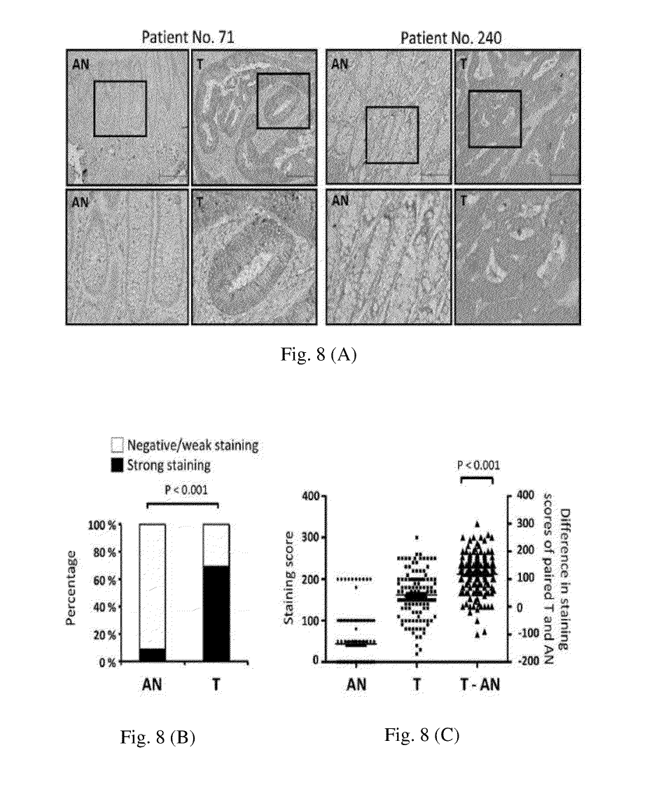

In order to understand pGSN expression in CRC, the Inventors use the anti-gelsolin N20 antibody to perform IHC (immunohistochemistry) experiment in cancer tissues. The experimental results show that pGSN does not express or only slightly expresses in the cells of the neighboring normal epithelial tissues but massively expresses in cells of cancer tissues. The representative stained images of the two groups of tissues are shown in FIG. 8A. The statistic analysis shows that pGSN massively and intensively expresses in the cells of cancer tissues in 68.9% (102/148) sections and that pGSN does not express or only slightly expresses in the cells of the neighboring normal epithelial tissues in 91.7% (122/148) sections, as shown in FIG. 8B. It is statistically significant that pGSN does not express in the normal epithelial tissues neighboring the CRC tissues but massively expresses in the CRC tissues in the 133 sections simultaneously having cancer tissues and normal epithelial tissues, as shown in FIG. 8C.

### Embodiment VII

### The Role of pGSN in Regulating the Cell Migration of CRC Cell Line

In order to investigate the role pGSN in distal CRC metastasis, the Inventors used CRC cell line SW480 to investigate whether pGSN takes part in cell migration of CRC cell line.

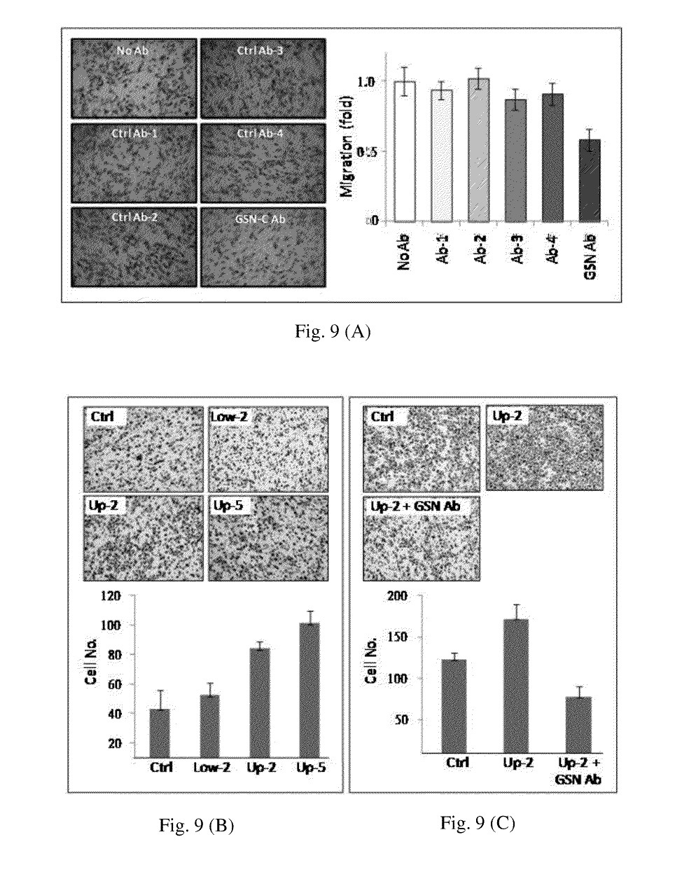

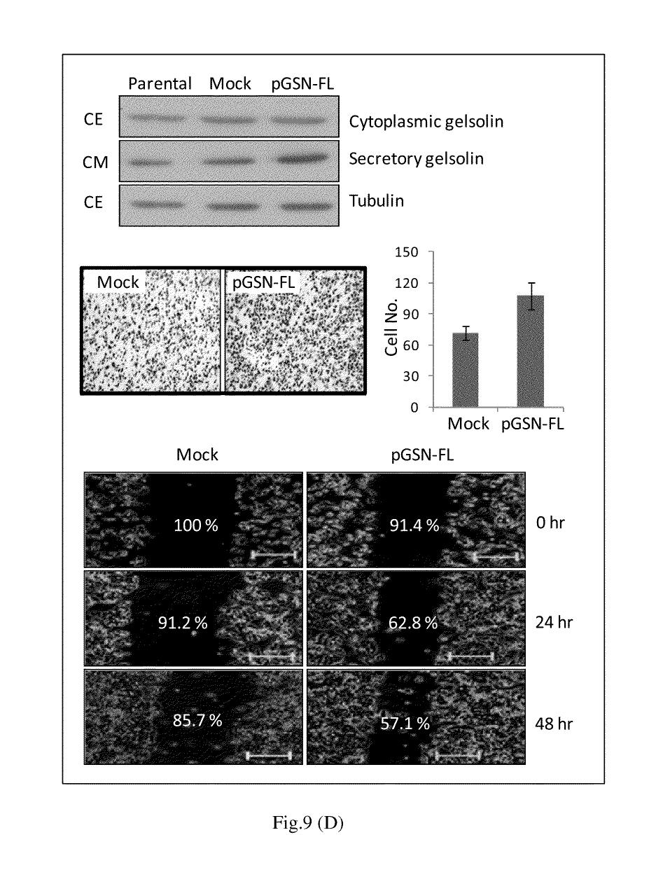

The Inventors combined an antibody with extracellular pGSN, and use the Transwell Assay to analyze mobility of cells. The experimental result show that the antibody antagonizes the extracellular pGSN secreted by cells and reduces cell mobility and addition of other antibodies would not influence cell mobility, as shown in FIG. 9A. The result implies that extracellular pGSN is associated with CRC cell migration.

Next, the Inventors added purified full-length pGSN recombinant protein to the upper or lower Transwell Assay chamber to undertake a Transwell migration assay so as to verify whether pGSN is a chemoattractant or a regulatory molecule for CRC cell migration. In the experiment, take 2×10⁵ cells from CRC cell line (SW480 or SW620), suspend the cells in a serum-free medium, and place the medium in upper chambers of the Transwell migration module (BD Bioscience). Next, add to the lower chambers several doses of L-15 medium (Invitrogen, Carlsbad, Calif., USA), which all contain 10% FCS and respectively contain no antibody (No Ab), 1 μg of anti-gelsolin N20 antibody plus 1 of anti-gelsolin monoclonal antibody, and control polyclonal antibodies Ab-1 to Ab-4 each weighing 2 μg, until each lower chamber is filled with 600 μl of medium. Let cell migration proceed for 16 hours. Next, fix the attaching cells, stain the attaching cells with Giemsa (Sigma), and use cotton swabs to remove the cells that do not migrate from the upper chambers to the lower chambers. Next, select 9 sections under a microscope at a magnification of 200× to count the numbers of the cells having passed the diaphragm and reached the lower surface of the filter film.

From the experimental results shown in FIG. 9B, it is found: while the purified full-length pGSN recombinant protein (2 μg/ml) is not added to the lower layer but added to the upper layer, cell mobility is obviously increased. It indicates that the purified full-length pGSN recombinant protein is not a chemoattractant but an extracellular regulation factor of CRC cell migration.

Refer to FIG. 9C. While the full-length pGSN recombinant protein is antagonized by the antibody, the capability of the full-length pGSN recombinant protein to induce cell migration is reduced. It indicates that pGSN is an extracellular regulatory protein associated with cellular migration of CRC cell line. Besides, the Inventors transfected the pGSN-bearing expression plasmid to the CRC cell line (SW480) and found that pGSN increases obviously in the culture medium but the amounts of non-pGSN gelsolin (i.e. cytoplasmic gelsolin) keep unchanged. The Inventors analyzed the cell migration of the transfected cells and found that pGSN expresses massively in the SW480 cells could drastically enhance the cell's migration ability in the Transwell assay and the Wound healing assay, as shown in the middle and lower illustrations of FIG. 9D. Therefore, pGSN is indeed an important extracellular modulator for CRC cell migration and can be used as a blood biomarker for detecting CRC metastasis.

The characteristics disclosed in the specification can be combined in any way without departing from the spirit of the present invention. The characteristics disclosed in the specification can be replaced by substitute characteristics having identical, equivalent or similar functions without departing from the spirit of the present invention. Therefore, the characteristic disclosed in the specification is only an exemplification of a group of characteristics having identical, equivalent or similar functions unless it is stated otherwise.

According to the specification, the persons skilled in the art should be able to modify or vary the characteristic of the present invention without departing from the spirit of the present invention. Therefore, any equivalent modification or variation according to the spirit of the present invention is to be also included within the scope of the present invention.

What is claimed is:

1. A plasma biomarker for evaluating distal metastasis of colorectal cancer, comprising at least one secretory gelsolin (pGSN).

2. The plasma biomarker for evaluating distal metastasis of colorectal cancer according to claim 1, wherein said secretory gelsolin comprises an amino acid sequence with similarity of more than 95% to SEQ ID NO:1.

3. The plasma biomarker for evaluating distal metastasis of colorectal cancer according to claim 1, further comprising at least one existing plasma biomarker for evaluating distal metastasis of colorectal cancer.

4. The plasma biomarker for evaluating distal metastasis of colorectal cancer according to claim 3, wherein said existing plasma biomarker for evaluating distal metastasis of colorectal cancer is a carcinoembryonic antigen (CEA).

5. The plasma biomarker for evaluating distal metastasis of colorectal cancer according to any one of claim 1 to 4, which is used to evaluate distal metastasis of colorectal cancer in cooperation with an ELISA (enzyme-linked immunosorbent assay) method, a bead-based immunoassay method, a mass spectrometry-based assay method, or a method using combination of immunoassay and mass spectrometry-based assay.

6. A method for evaluating distal metastasis of colorectal cancer, comprising steps of:
(1) sampling: obtaining a blood sample from a testee;
(2) detecting: detecting whether said blood sample has at least on plasma biomarker, including secretory gelsolin (pGSN); and
(3) analyzing: using a standard cure to calculate a concentration of said plasma biomarker in said blood sample, and comparing said concentration with a concentration of said plasma biomarker of a healthy person.

7. The method for evaluating distal metastasis of colorectal cancer according to claim 6, wherein said plasma biomarker includes an existing plasma biomarker for evaluating distal metastasis of colorectal cancer.

8. The method for evaluating distal metastasis of colorectal cancer according to claim 7, wherein said existing plasma biomarker for evaluating distal metastasis of colorectal cancer is a carcinoembryonic antigen (CEA).

9. The method for evaluating distal metastasis of colorectal cancer according to any one of claim 6 to 8, wherein said blood sample is a whole blood sample, a serum sample, or a plasma sample.

10. The method for evaluating distal metastasis of colorectal cancer according to claim 6, wherein said secretory gelsolin comprises an amino acid sequence with similarity of more than 95% to SEQ ID NO: 1.

11. The method for evaluating distal metastasis of colorectal cancer according to claim 6, wherein said step of sampling uses an ELISA (enzyme-linked immunosorbent assay) method, a bead-based immunoassay method, a mass spectrometry-based assay method, or method using combination of immunoassay and mass spectrometry-based assay to detect said plasma biomarker in said blood sample.

12. The method for evaluating distal metastasis of colorectal cancer according to claim 6, wherein said step of sampling uses an antibody specifically recognizing said secretory gelsolin to detect said secretory gelsolin in said blood sample.

13. The method for evaluating distal metastasis of colorectal cancer according to claim 12, wherein said antibody specifically recognizing said secretory gelsolin is fabricated via using a peptide comprising an amino acid sequence of SEQ ID NO:2 as an antigen.

14. The method for evaluating distal metastasis of colorectal cancer according to claim 13, wherein said antibody specifically recognizing said secretory gelsolin is a monoclonal antibody, a polyclonal antibody, or a single chain antibody.

15. A kit for evaluating distal metastasis of colorectal cancer, comprising an antibody specifically recognizing secretory gelsolin.

16. The kit for evaluating distal metastasis of colorectal cancer according to claim 15, wherein said antibody specifically recognizing said secretory gelsolin binds to a protein comprising an amino acid sequence of SEQ ID NO:1.

17. The kit for evaluating distal metastasis of colorectal cancer according to claim 15, wherein said antibody specifically recognizing said secretory gelsolin is fabricated via using a peptide comprising an amino acid sequence of SEQ ID NO:2 as an antigen.

18. The kit for evaluating distal metastasis of colorectal cancer according to claim 15, wherein said antibody specifically recognizing said secretory gelsolin is a monoclonal antibody, a polyclonal antibody, or a single chain antibody.
